(19) United States
(12) Patent Application Publication
(10) Pub. No.: US 2026/0130894 A1
(43) Pub. Date: May 14, 2026

(54) COMPOSITION COMPRISING ISOQUINOLINE DERIVATIVE AS ACTIVE INGREDIENT FOR PREVENTION OR TREATMENT OF PARKINSON'S DISEASE

(71) Applicants: ALTMEDICAL CO., LTD., Seoul (KR); DONG-A UNIVERSITY RESEARCH FOUNDATION FOR INDUSTRY-ACADEMY COOPERATION, Busan (KR)

(72) Inventors: Jeanho YUN, Busan (KR); Jong Hyun CHO, Gyeongsangnam-do (KR); Jee-Hyun UM, Busan (KR); Dong Jin SHIN, Busan (KR); Young Yeon KIM, Busan (KR); Se Myeong CHOI, Busan (KR)

(21) Appl. No.: 19/120,024

(22) PCT Filed: Oct. 10, 2023
(86) PCT No.: PCT/KR2023/015548

§ 371 Date: Apr. 10, 2025

(30) Foreign Application Priority Data
Oct. 11, 2022 (KR) 10-2022-0129937

(51) Int. Cl. A61K 31/4375; A61P 25/16

(52) U.S. Cl. CPC A61K 31/4375; A61P 25/16

(57) ABSTRACT

The present invention relates to a pharmaceutical composition for the prevention or treatment of Parkinson's disease. Leading to the present invention, an isoquinoline derivative, identified through screening based on mitophagy activation, was found to exhibit an excellent effect in promoting mitophagy and proved to be a fundamental therapeutic agent for Parkinson's disease. Specifically, the isoquinoline derivative according to the present invention not only reduces mitochondrial dysfunction closely associated with Parkinson's disease in a Parkinson' disease animal model but also ameliorates motor function impairment in the Parkinson's disease animal model. Particularly, in the Parkinson's disease animal model treated with the isoquinoline derivative according to the present invention, the demise of dopaminergic neurons, a primary cause of Parkinson's disease, was effectively inhibited. Thus, the isoquinoline derivative according to the present invention is expected to be advantageously utilized as a fundamental therapeutic agent in the field of prevention, alleviation, and/or treatment of Parkinson's disease.

### TECHNICAL FIELD

The present invention relates to uses of a novel isoquinoline derivative to prevent, improve, and/or treat Parkinson's disease.

This application claims priority to and the benefit of Korean Patent Application No. 10-2022-0129937, filed on Oct. 11, 2022, the disclosure of which is incorporated herein by reference in its entirety.

### BACKGROUND ART

Parkinson's disease is one of the three major senile neurodegenerative diseases. It occurs due to a decrease in dopamine production and action caused by the loss of dopaminergic neurons present in the substantia nigra of midbrain, and in severe cases, it may develop to dementia. The incidence of Parkinson's disease is steadily increasing worldwide, and particularly, a clear increase in incidence in the elderly is shown, so it is urgent to develop therapeutic agents targeting the cause of the disease. Particularly, there is a need for the development of therapeutic agents that are capable of fundamental treatment of the disease. However, currently, there are no effective treatments or therapeutic agents for Parkinson's disease other than symptom relief agents (levodopa, dopamine agonists, MAO-B, etc.), which can be used in the early stage of the disease as a therapeutic agent. Levodopa can relieve symptoms by restoring reduced dopamine, but it does not address the underlying cause of Parkinson's disease, an increase in the required dosage increases over time, and some patients experience serious side effects such as dyskinesia and hallucinations. Recently, attempts have been made to develop treatments for Parkinson's disease based on stem cells, genes, and antibodies, but no therapeutic agent with a proven successful therapeutic effect has yet been discovered.

Meanwhile, mitophagy is an intracellular degradation mechanism that removes damaged or unnecessary mitochondria. When mitochondria are damaged, they are surrounded by a membrane to form an autophagosome, which is then fused with a lysosome to selectively remove the damaged mitochondria. It is known that the activity of the mitophagy is important for regulating mitochondria function in various cells including neurons and maintaining tissue functions. In addition, mitophagy in neurons has been reported to have a protective effect against various stresses and to be important for resistance to neurodegeneration. Recently, there has been increasing interest in the link between mitochondrial dysfunction and dopaminergic neuron death. For example, a decrease in mitophagy activity in hereditary and sporadic Parkinson's disease has been observed. In addition, neurotoxins such as rotenone and paraquat, which cause Parkinson's disease, act as mitochondria inhibitors, and it has been found that mutations in Parkinson's disease-associated genes, including PINK1 and Parkin, can also cause mitochondrial dysfunction. In addition, alpha-synuclein, known to play an important role in the development of Parkinson's disease, has been found to accumulate in mitochondria and cause mitochondrial dysfunction. Various mitochondrial dysfunctions have been confirmed in dopaminergic neurons of actual patients with Parkinson's disease. These results imply that mitochondrial dysfunction is an important factor in the occurrence and development of Parkinson's disease. However, there is currently no development of therapeutic agents for Parkinson's disease that target improvement in mitochondrial dysfunction.

In order to discover a mitophagy regulator that can exert a disease treatment effect by promoting mitophagy activity, it is essential to have a research system that can easily measure mitophagy changes in cells and in vivo. However, due to the absence of experimental methods for sensitively and quantitatively measuring mitophagy activity, searching for a mitophagy regulating compound using mitophagy activity itself as a read-out has not been performed. The LC3-based detection method that has been widely used so far has limitations in that it can only measure an autophagosome-forming stage, which is the initial stage of mitophagy, and therefore has low measurement sensitivity and difficulty in quantitative measurement. In addition, most attempts to discover control materials in several research groups have also failed to discover materials that can control mitophagy activity under actual physiological conditions because they used indirect read-out indicators such as the mitochondrial movement of PINK-Parkin or the mitochondrial fission that occurs during mitophagy.

Currently, experimental methods to induce mitophagy activity involve treating with so-called ‘mitochondrial toxins’, such as CCCP, FCCP, and rotenone, which induce mitochondrial dysfunction. However, the CCCP and FCCP depolarize the mitochondrial membrane potential as a mitochondrial membrane potential inhibitors (uncouplers), and rotenone acts as a Complex I inhibitor. These mitochondrial toxins induce mitophagy activity by directly causing mitochondrial damage, thereby activating the mechanism for removing damaged mitochondria. However, their strong toxicity to cells limits their use as drugs to promote mitophagy activity.

### DISCLOSURE

### Technical Problem

The present invention has been devised to address the above-described problems, and was completed by confirming that an isoquinoline derivative discovered by screening based on mitophagy activity exhibits an excellent mitophagy-promoting effect, and thus can be utilized as a fundamental therapeutic agent for Parkinson's disease.

Therefore, the present invention is directed to providing a pharmaceutical composition for preventing or treating Parkinson's disease, which includes an isoquinoline derivative or a pharmaceutically acceptable salt thereof as an active ingredient.

The present invention is also directed to providing a kit for preventing or treating Parkinson's disease, which includes the pharmaceutical composition.

The present invention is also directed to providing a food composition for preventing or improving Parkinson's disease, which includes an isoquinoline derivative or a sitologically acceptable salt thereof as an active ingredient.

However, technical problems to be solved in the present invention are not limited to the above-described problems, and other problems which are not described herein will be fully understood by those of ordinary skill in the art from the following descriptions.

### Technical Solution

The present invention provides a pharmaceutical composition for preventing or treating Parkinson's disease, which includes an isoquinoline derivative represented by Chemical Formula 1 below or a pharmaceutically acceptable salt thereof as an active ingredient.

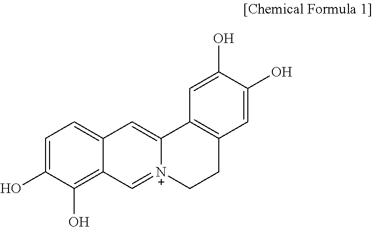 [SMILES: Oc1cc2c(cc1O)-c1cc3ccc(O)c(O)c3c[n+]1CC2]

The present invention also provides a method of preventing or treating Parkinson's disease, which includes administering the isoquinoline derivative represented by Chemical Formula 1 or a pharmaceutically acceptable salt thereof to a subject in need thereof.

The present invention also provides a use of the isoquinoline derivative represented by Chemical Formula 1 or a pharmaceutically acceptable salt thereof to prevent or treat Parkinson's disease.

The present invention also provides a use of the isoquinoline derivative represented by Chemical Formula 1 or a pharmaceutically acceptable salt thereof to prepare a drug for preventing or treating Parkinson's disease.

The present invention also provides a kit for preventing or treating Parkinson's disease, which includes the isoquinoline derivative represented by Chemical Formula 1, a pharmaceutically acceptable salt thereof, or the composition.

The present invention also provides a food composition for preventing or improving Parkinson's disease, which includes the isoquinoline derivative represented by Chemical Formula 1 or a sitologically acceptable salt thereof as an active ingredient.

In one embodiment of the present invention, the isoquinoline derivative or a pharmaceutically acceptable salt thereof may promote mitophagy activity, but the present invention is not limited thereto.

In another embodiment of the present invention, the isoquinoline derivative or a pharmaceutically acceptable salt thereof may satisfy, but is not limited to, one or more selected from the group consisting of.
(a) reducing the membrane potential of mitochondria;
(b) reducing the reactive oxygen species level in mitochondria; and
(c) increasing the ATP synthesis capacity of mitochondria.

In still another embodiment of the present invention, the isoquinoline derivative or a pharmaceutically acceptable salt thereof may increase the number or activity of dopaminergic neurons, but the present invention is not limited thereto.

In yet another embodiment of the present invention, the Parkinson's disease may be hereditary Parkinson's disease or sporadic Parkinson's disease, but the present invention is not limited thereto.

### Advantageous Effects

The present invention relates to a pharmaceutical composition for preventing or treating Parkinson's disease, and was completed by confirming that an isoquinoline derivative discovered through screening based on mitophagy activity exhibits excellent mitophagy-promoting effects, and thus can be utilized as a fundamental treatment for Parkinson's disease. Specifically, it was confirmed that the isoquinoline derivative according to the present invention can not only improve mitochondrial dysfunction closely related to Parkinson's disease in an animal model of Parkinson's disease, but also improve impaired motor function in an animal model of Parkinson's disease. Particularly, in the animal model of Parkinson's disease treated with the isoquinoline derivative according to the present invention, dopaminergic neuron death, which is the main cause of Parkinson's disease, appears to be effectively inhibited. Therefore, the isoquinoline derivative according to the present invention is expected to be useful in the fields of prevention, improvement, and/or treatment of Parkinson's disease as a fundamental therapeutic agent for the disease.

### BRIEF DESCRIPTION OF THE DRAWINGS

FIGS. 1A to 1C show the results of analyzing the promoting effect of mitophagy activity by the isoquinoline derivative compound according to an embodiment of the present invention (referred to as “CD1-012,” hereinafter the same) in a normal human lung cell line (FIG. 1A, FACS results; FIG. 1B, confocal microscopy observation results; and FIG. 1C, quantitative measurement of mitochondrial changes using mito-YFP fluorescent protein).

FIGS. 2A and 2B show the results of analyzing the promoting effect of mitophagy activity by CD1-012 in a SH-SY5Y cell line (FIG. 2A) and a Hela-Parkin cell line (FIG. 2B).

FIGS. 3A and 3B show the results of analyzing the mitophagy activity in BEAS-2B cells according to the treatment concentration (FIG. 3A) and treatment time (FIG. 3B) of CD1-012.

FIGS. 4A and 4B show the results of confirming changes in mitophagy activity (FIG. 4A) and autophagy activity (FIG. 4B) in cells treated with CD1-012.

FIG. 5 shows the result of comparing the promoting effect of mitophagy activity by CD1-012 compared with palmatine and berberine, according to concentration.

FIG. 6 shows the result of analyzing the levels of mitochondrial membrane potential and mitochondrial reactive oxygen species after treating cells with CD1-012 or the control compound CCCP.

FIG. 7 shows result of analyzing mitophagy promoting activity of CD1-012 and the control compound CCCP in PINK1 knockdown cell lines (shPINK1).

FIG. 8A shows result of measuring the levels of the mitochondrial protein Cox2 after treating wild-type (WT) and PINK1-deficient (PINK1^{-/-}) mouse fibroblast cell lines with CD1-012 at different concentrations to verify its mitophagy-promoting activity.

FIG. 8B shows the analysis of mitophagy activity after treating human lung cell lines with CD1-012 following the suppression of PINK1 expression (shPINK1) to determine whether the mitophagy-promoting activity of CD1-012 is dependent on the PINK1 gene.

FIG. 8C shows the results of CD1-012 treatment restoring the mitochondrial membrane potential that was reduced in PINK1-deficient cells.

FIG. 8D shows the results of CD1-012 treatment reducing the level of reactive oxygen species (ROS) in mitochondria, which was increased in PINK1-deficient cells.

FIG. 8E shows the results of CD1-012 treatment restoring the mitochondrial ATP production, which was reduced in PINK1-deficient cells.

FIG. 9A shows the results of analyzing the morphology of mitochondria in dopaminergic neurons of brain tissue (left) and measuring the number of abnormal mitochondria (right) after treating Drosophila models of Parkinson's disease (B9) with CD1-012 to confirm the effect of CD1-012 on improving mitochondrial function.

FIGS. 9B and 9C show the results of confirming the ROS levels (FIG. 9B) and ATP production capacity (FIG. 9C) of mitochondria in brain tissue after treating Drosophila models of Parkinson's disease with CD1-012.

FIGS. 10A to 10D show the results of measuring mitochondrial membrane potentials (FIGS. 10A and 10B) and ATP production capacity (FIGS. 10C and 1OD) after treating cellular models of sporadic Parkinson's disease, produced by treatment of MPP⁺ (FIGS. 10A and 10C) or 60HDA (FIGS. 10B and 10D), with CD1-012.

FIG. 11A is an animal experimental schedule to confirm the therapeutic effect of CD1-012 on impaired motor performance caused by Parkinson's disease (MPTP, Parkinson's disease-inducing material).

FIGS. 11B and 11C show the results of confirming the motor performance of animal models of Parkinson's disease treated with CD1-012 using a pole test (FIG. 11B) and a Rota-rod test (FIG. 11C).

FIGS. 12A and 12B show the results of measuring the membrane potentials (FIG. 12A) and ATP production capacity (FIG. 12B) of mitochondria in the substantia nigra of mouse in animal models of Parkinson's disease treated with CD1-012.

FIGS. 13A and 13B show results of observing stained dopaminergic neurons (FIG. 13A) and measuring the number of dopaminergic neurons (FIG. 13B) in the substantia nigra of mouse in animal models of Parkinson's disease treated with CD1-012.

### BEST MODES OF THE INVENTION

The present invention relates to a pharmaceutical composition for preventing or treating Parkinson's disease, and was completed by confirming that an isoquinoline derivative discovered by screening based on mitophagy activity exhibits an excellent mitophagy-promoting effect, and thus can be utilized as a fundamental therapeutic agent for Parkinson's disease.

Specifically, the compound according to the present invention is an isoquinoline derivative with an identified mitophagy-specific promoting function and was confirmed to have no mitochondrial toxicity or cytotoxicity, and it has been confirmed through previous studies that it can improve mitochondrial dysfunction in an Alzheimer's disease model.

To analyze the therapeutic effect of the isoquinoline derivative on Parkinson's disease, the present inventors treated a Parkinson's disease gene PINK1-deficient cell line and a PINK1-deficient Drosophila with the compound, and confirmed that a mitochondrial function is improved (Examples 5 and 6).

In addition, when neural cell lines that were treated with 1-methyl-4-phenylpyridinium (MPP*) or 6-hydroxydopamine (6-OHDA), which is a neurotoxin that causes Parkinson's disease, were treated with the compound of the present invention, mitochondrial dysfunction was improved, verifying the therapeutic effect of the compound of the present invention on sporadic Parkinson's disease (Example 7).

In addition, it was confirmed that, as a result of treating an animal model of Parkinson's disease manufactured by injecting the neurotoxin, 1-methyl-4-phenyl-1,2,3,6-tetrahydropyridine (MPTP), with the compound of the present invention, motor performance impairment is improved (Example 8), and it was confirmed that not only was the mitochondrial dysfunction in the substantia nigra tissue of the mouse model improved, but also dopaminergic neuron death of Parkinson's disease was inhibited (Example 9).

Accordingly, the isoquinoline derivative according to the present invention can not only improve the main symptoms (mitochondrial dysfunction and impaired motor function) of Parkinson's disease, but also inhibit the fundamental pathogenic mechanism by promoting mitophagy. Thus, it can be used as an effective therapeutic agent for Parkinson's disease.

Hereinafter, the present invention will be described in detail.

The present invention is directed to providing a pharmaceutical composition for preventing or treating Parkinson's disease, which includes an isoquinoline derivative represented by Chemical Formula 1 below or a pharmaceutically acceptable salt thereof as an active ingredient.

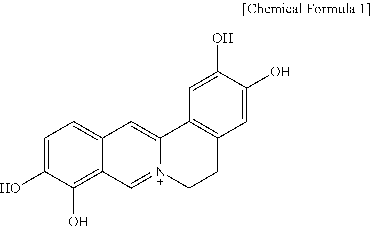 [SMILES: Oc1cc2c(cc1O)-c1cc3ccc(O)c(O)c3c[n+]1CC2]

In the present invention, the term “pharmaceutically acceptable salt” comprises a salt derived from pharmaceutically acceptable inorganic acids, organic acids, or bases.

The term “pharmaceutically acceptable” as used in the disclosure refers to a compound or composition that is suitable for use in contact with the tissues of a subject (e.g., a human) with a reasonable benefit/risk ratio, without excessive toxicity, irritation, allergic reaction, or other issues or complications, and that falls within the scope of sound medical judgment.

Examples of suitable acids include hydrochloric acid, bromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, gluconic acid, naphthalene-2-sulfonic acid, benzene sulfonic acid, etc. Acid addition salts may be manufactured by conventional methods, for example, by dissolving the compound in an excess of an acid aqueous solution and precipitating this salt using a water-miscible organic solvent such as methanol, ethanol, acetone, or acetonitrile. Acid addition salts may also be prepared by heating an equimolar amount of the compound and an acid or alcohol in water and subsequently evaporating the mixture to dryness, or by aspiration-filtration of the precipitated salt.

Salts derived from suitable bases may include alkali metals such as sodium, potassium, alkaline earth metals such as magnesium, and ammonium, but are not limited thereto. Alkali metal or alkaline earth metal salts, for example, may be obtained by dissolving the compound in an excess of alkali metal hydroxide or alkaline earth metal hydroxide solution, filtering off the insoluble compound salt, and then evaporating and drying the filtrate. Here, it is pharmaceutically suitable to manufacture particularly sodium, potassium, or calcium salts as the metal salts, and the corresponding silver salts may be obtained by reacting an alkali metal or alkaline earth metal salt with a suitable silver salt (for example, silver nitrate).

The scope of the compounds of the present invention includes not only pharmaceutically acceptable salts but also all isomers, hydrates, and solvates that can be prepared by conventional methods.

In one embodiment of the present invention, the isoquinoline derivative represented by Chemical Formula 1 may be prepared as shown in Reaction Scheme 1 below according to a preparation method that includes adding palmatine represented by Chemical Formula 2 or berberine represented by Chemical Formula 3 and a Lewis acid catalyst to an organic solvent to allow a reaction (Step 1).

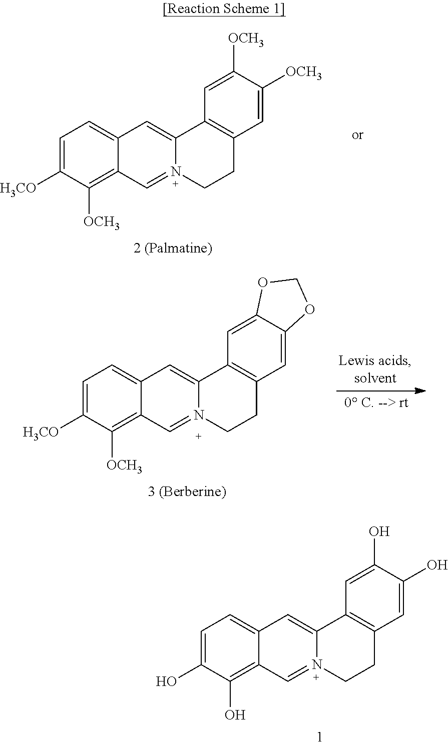

According to an embodiment of the present invention, the isoquinoline derivative or a pharmaceutically acceptable salt thereof may be a derivative in which a hydrophobic substituent (methoxy group) in the core structure of the palmatine or berberine is substituted with a hydrophilic substituent or a functional group that can provide a hydrogen bond between molecules (hydroxyl group). For example, the isoquinoline derivative according to the present invention may be selected from 2,3,5,10-tetrahydroxy-5,6-dihydroisoquinolino[3,2-a]isoquinolin-7-ium bromide (2,3,9,10-tetrahydroxy-5,6-dihydroisoquinolino[3,2-a]isoquinolin-7-ium bromide), 2,3,5,10-tetrahydroxy-5,6-dihydroisoquinolino[3,2-a]isoquinolin-7-ium hydroxide (2,3,9,10-tetrahydroxy-5,6-dihydroisoquinolino[3,2-a]isoquinolin-7-ium hydroxide), and 2,3,5,10-tetrahydroxy-5,6-dihydroisoquinolino[3,2-a]isoquinolin-7-ium chloride (2,3,9,10-tetrahydroxy-5,6-dihydroisoquinolino[3,2-a]isoquinolin-7-ium chloride), which are represented by Chemical Formulas 1a to 1c, respectively. In the disclosure, the 2,3,5,10-yetrahydroxy-5,6-dihydroisoquinolino[3,2-a]isoquinolin-7-ium bromide may be referred to as “CD1-012.”

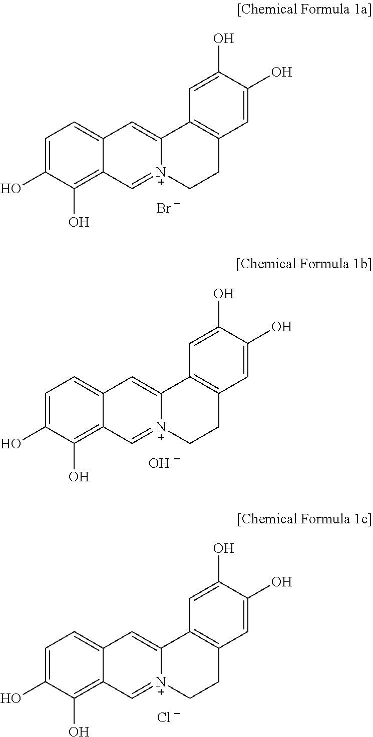

The isoquinoline derivative according to the present invention or a pharmaceutically acceptable salt thereof is characterized by promoting mitophagy activity (i.e., activating mitophagy).

In the present invention, the term “mitophagy” refers to an intracellular degradation mechanism which removes damaged or unnecessary mitochondria. Mitophagy is a process in which autophagosomes are formed when mitochondria are damaged, and fused with lysosomes to selectively degrade and remove the damaged mitochondria. Mitophagy is a distinct mechanism from autophagy, which is a mechanism that decomposes and recycles unnecessary components within cells (old proteins, protein aggregates, organelles, pathogens that have invaded cells, etc.) to produce a macromolecular precursor and energy when the cells are under nutrient deficiency. Mitophagy is regulated independently of regulatory signals such as nutrients, energy, and stress, which control autophagy.

The present inventors confirmed according to an exemplary embodiment that the isoquinoline derivative according to the present invention exhibits a higher mitophagy activating effect than other known mitophagy promoters, thereby achieving a therapeutic effect for Parkinson's disease. Therefore, the isoquinoline derivative according to the present invention or a pharmaceutically acceptable salt thereof may exhibit an excellent therapeutic effect particularly in a patient with Parkinson's disease having a decreased mitophagy level (e.g., a mitophagy level of neurons). For example, the compound according to the present invention may exhibit an excellent therapeutic effect particularly in a patient with Parkinson's disease having mutations of PINK1 gene and/or protein. The mutations include a decrease in the level and/or activity of the PINK1 protein. However, the present inventors have confirmed through exemplary embodiments that the compound can promote mitophagy independently of the PINK1-Parkin pathway, and it is clear that the effect of the compound of the present invention in preventing and/or treating Parkinson's disease is not limited to a patient with the mutation(s).

In addition, the isoquinoline derivative according to the present invention or a pharmaceutically acceptable salt thereof may satisfy one or more properties selected from the group consisting of:
(a) reducing the membrane potential of mitochondria;
(b) reducing the reactive oxygen species (ROS) level in mitochondria; and
(c) increasing the ATP synthesis capacity of mitochondria.

That is, the isoquinoline derivative according to the present invention or a pharmaceutically acceptable salt thereof may raise the level and/or activity (function) of mitochondria. The effect of the compound according to the present invention on improving the mitochondrial level and/or activity may be achieved by the mitophagy activating effect of the compound. Accordingly, the isoquinoline derivative according to the present invention or a pharmaceutically acceptable salt thereof may exhibit an excellent therapeutic effect particularly in a patient with Parkinson's disease having the decreased mitochondrial level and/or function (e.g., the level and/or function of mitochondria in neurons).

In addition, the isoquinoline derivative according to the present invention or a pharmaceutically acceptable salt thereof may increase the number and/or function of dopaminergic neurons.

In the present invention, “dopamine” is an organic compound of the catecholamine series, and a neurotransmitter found in the central nervous system of various animals. Dopamine is produced in several areas of the brain, including the substantia nigra and the spinal tegmentum. “Dopaminergic neurons” are the main source of dopamine, and are cells that play an important role in controlling various brain functions such as voluntary movement, mood, addiction, stress, etc. Particularly, a decrease in the number of dopaminergic neurons due to their death is known to be associated with Parkinson's disease. According to exemplary embodiments, the present inventors confirmed that the compound according to the present invention inhibits dopaminergic neuron death in the substantia nigra, thereby contributing to the restoration of the numerical decrease in dopaminergic neurons.

In the present invention, “Parkinson's disease” is a progressive neurodegenerative disease characterized by parkinsonian symptoms such as low movement, resting tremors, muscle stiffness, a shuffling gait, and stooped posture. This disease is mainly caused by decreased stimulation of the motor neuron cortex due to imperfect dopamine production and action in the substantia nigra. In the present invention, Parkinson's disease includes hereditary Parkinson's disease caused by genetic factors (e.g., inheritance of specific mutated genes, etc.) and sporadic Parkinson's disease caused by mutations in various genes and other causes that have not been identified. Sporadic Parkinson's disease accounts for 80 to 90% of Parkinson's disease patients, but its pathogenesis has not yet been elucidated. However, reduced mitophagy activity has been observed in both sporadic Parkinson's disease and hereditary Parkinson's disease.

The content of the isoquinoline derivative or pharmaceutically acceptable salt thereof in the composition of the present invention may be appropriately adjusted depending on the symptoms of a disease, the degree of progression of symptoms, the condition of a patient, and the like, and may range from, for example, 0.0001 wt % to 99.9 wt % or 0.001 wt % to 50 wt % with respect to a total weight of the composition, but the present invention is not limited thereto. The amount ratio is a value based on the amount of dried product from which a solvent is removed.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The present invention provides a pharmaceutical composition for preventing or treating Parkinson's disease, which includes an isoquinoline derivative represented by Chemical Formula 1 below or a pharmaceutically acceptable salt thereof as an active ingredient.

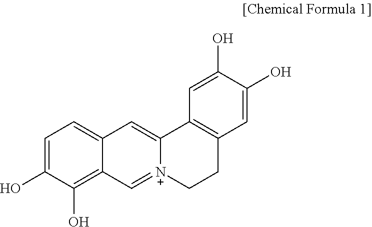 [SMILES: Oc1cc2c(cc1O)-c1cc3ccc(O)c(O)c3c[n+]1CC2]

In one embodiment of the present invention, the isoquinoline derivative or a pharmaceutically acceptable salt thereof may specifically promote mitophagy activity, but the present invention is not limited thereto.

In one embodiment of the present invention, the mitophagy activity may be independent of PINK1, but the present invention is not limited thereto.

In one embodiment of the present invention, the isoquinoline derivative or a pharmaceutically acceptable salt thereof may increase the number or activity of dopaminergic neurons, but the present invention is not limited thereto.

In one embodiment of the present invention, the Parkinson's disease may be hereditary Parkinson's disease or sporadic Parkinson's disease, but the present invention is not limited thereto.

In one embodiment of the present invention, the isoquinoline derivative or a pharmaceutically acceptable salt thereof may be contained at more than 0 to 40 μM, but the present invention is not limited thereto.

In one embodiment of the present invention, the isoquinoline derivative or a pharmaceutically acceptable salt thereof may improve mitochondrial dysfunction, but the present invention is not limited thereto.

In one embodiment of the present invention, the improvement in mitochondrial dysfunction may be, but is not limited to, any one selected from the group consisting of:
(a) maintaining the normal membrane potential level in mitochondria;
(b) reducing the reactive oxygen species (ROS) level in mitochondria; and
(c) increasing the ATP synthesis capacity of mitochondria.

The present invention provides a kit for preventing or treating Parkinson's disease, which includes: the isoquinoline derivative or a pharmaceutically acceptable salt thereof, or a pharmaceutical composition including the same as an active ingredient; and instructions.

The present invention provides a food composition for preventing or improving Parkinson's disease, which includes an isoquinoline derivative represented by Chemical Formula 1 below or a sitologically acceptable salt thereof as an active ingredient.

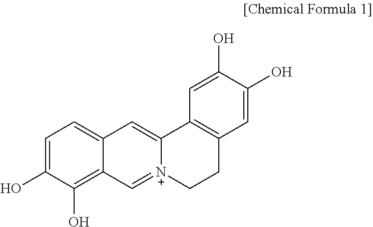 [SMILES: Oc1cc2c(cc1O)-c1cc3ccc(O)c(O)c3c[n+]1CC2]

In one embodiment of the present invention, the Parkinson's disease may be hereditary Parkinson's disease or sporadic Parkinson's disease, but the present invention is not limited thereto.

The present invention provides a method of preventing or treating Parkinson's disease, which includes administering the isoquinoline derivative represented by Chemical Formula 1 or a pharmaceutically acceptable salt thereof to a subject in need thereof.

The present invention provides a use of the isoquinoline derivative represented by Chemical Formula 1 or a pharmaceutically acceptable salt thereof to prevent or treat Parkinson's disease.

The present invention provides a use of the isoquinoline derivative represented by Chemical Formula 1 or a pharmaceutically acceptable salt thereof for the manufacture of a medicament for preventing or treating Parkinson's disease.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan(propylene glycol monostearate), glycerin, Adeps solidus, buytyrum Tego-G, cebes Pharma 16, hexalide base 95, cotomar, Hydrokote SP, S-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types (AB, B, A, BC, BBG, E, BGF, C, D, 299), suppostal (N, Es), Wecoby (W, R, S, M, Fs), and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined by the type of drug that is the active ingredient, along with other relevant factors such as the disease to be treated, the route of administration, and the patient's age, gender, weight, and severity of the disease. Specifically, the effective amount of the composition according to the present invention may vary depending on the age, gender, and weight of the patient. Generally, it may be administered daily or every other day at a dose of 0.001 to 150 mg per 1 kg of body weight, preferably 0.01 to 100 mg per 1 kg of body weight, or divided into one to three doses per day. However, the dosage may be increased or decreased depending on the route of administration, severity of the disease, gender, weight, and age, and therefore, the above dosage range should not be construed as limiting the scope of the present invention in any way.

As used herein, the “subject” refers to a subject in need of treatment of a disease, and more specifically, refers to a mammal such as a human or a non-human primate, a mouse, a rat, a dog, a cat, a horse, and a cow, but the present invention is not limited thereto.

As used herein, the “administration” refers to providing a subject with a predetermined composition of the present invention by using an arbitrary appropriate method.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “improvement” as used herein means all actions that reduce the degree of parameters related to a target disease, e.g., symptoms via administration of the composition according to the present invention.

In addition, the present invention provides a kit for preventing or treating Parkinson's disease, which includes the isoquinoline derivative according to the present invention or a pharmaceutically acceptable salt thereof.

In the present invention, the term “kit” refers to a tool used for the prevention or treatment of Parkinson's disease, which includes the isoquinoline derivative of the present invention, a pharmaceutically acceptable salt thereof, or a composition including the same. The kit may further include other components, compositions, solutions, or devices that are conventionally required for the preparation, storage, or administration of the above substances, in addition to the compound or composition. For example, the kit may include an instruction manual describing the characteristics of the isoquinoline derivative according to the present invention or pharmaceutically acceptable salt thereof, as well as guidelines on proper use and storage.

Furthermore, the present invention provides a method for preparing the isoquinoline derivative or a pharmaceutically acceptable salt thereof, including a step of reacting palmatine or berberine with a Lewis acid catalyst in an organic solvent.

Specifically, the preparation method includes a step of reacting palmatine, represented by Chemical Formula 2, or berberine, represented by Chemical Formula 3, with a Lewis acid catalyst in an organic solvent, which can be represented by Reaction Scheme 1 below.

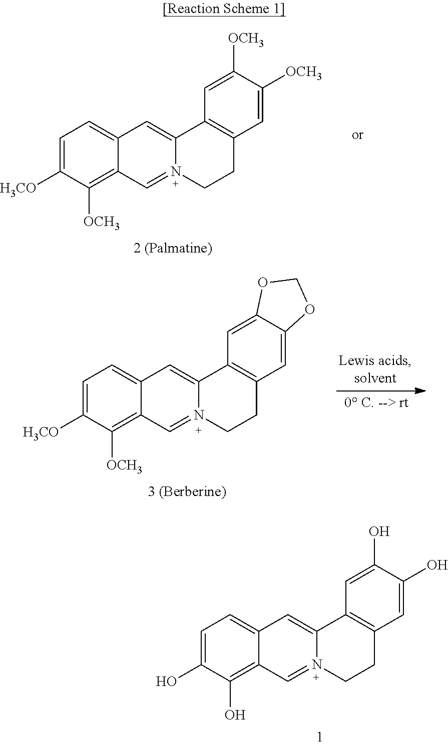

In one embodiment of the present invention, the Lewis acid catalyst may be at least one of metal halides such as BF₃, BBr₃, AlF₃, AlCl₃, AlBr₃, TiCl₄, TiBr₄, TiI₄, FeCl₃, FeCl₂, SnCl₂, SnCl₄, WCl₆, MoCl₅, SbCl₅, TeCl₂, and ZnCl₂; metal alkyl compounds such as Et₃Al, Et₂AlCl, EtAlCl₂, Et₃Al₂Cl₃, (i-Bu)₃Al, (i-Bu)₂AlCl, (i-Bu)AlCl₂, Me₄Sn, Et₄Sn, Bu₄Sn, and Bu₃SnCl; and metal alkoxy compounds such as Al(OR)₃₋ₓClₓ or Ti(OR)_{4-y}Cl_{y} (wherein the R represents an alkyl or aryl group, x is 1 or 2, y is an integer of 1 to 3), for example, a metal halide, for example, BBr₃, but is not limited thereto.

In one embodiment of the present invention, the organic solvent may be any one and more selected from the group consisting of dimethyl sulfoxide, dimethyl formamide, acetone, tetrahydrofuran, benzene, toluene, ether, methanol, hexane, cyclohexane, pyridine, acetic acid, carbon tetrachloride, chloroform, dichloromethane, and water, for example, dichloromethane, but is not limited thereto.

In one embodiment of the present invention, the addition of the Lewis acid catalyst may be performed in an inert gas. Specifically, the Lewis acid catalyst may be performed by using a method of adding to the palmatine or berberine dissolved in the organic solvent at about 0° C., in an inert gas atmosphere, for example, under a nitrogen stream.

In one embodiment of the present invention, after adding the Lewis acid catalyst, the reaction can be stirred for 10 to 14 hours, for example, 11 to 13 hours, for example, 12 hours, at room temperature, for example, 20° C. to 28° C., for example, 24° C. to 26° C., and the completion of the reaction may be confirmed, for example, using TLC (thin-layer Chromatography), but is not limited thereto.

Furthermore, the present invention provides a food composition for preventing or improving Parkinson's disease, which includes an isoquinoline derivative or a sitologically acceptable salt thereof. The food composition includes a health functional food composition.

In the present invention, the term “sitologically acceptable salt” comprises a salt derived from pharmaceutically acceptable inorganic acids, organic acids, or bases.

The isoquinoline derivative of present invention or a sitologically acceptable salt thereof may be used by adding the compound as is to food or may be used together with other foods or food ingredients, but may be appropriately used according to a typical method. The mixed amount of the active ingredient may be suitably determined depending on the purpose of use thereof (for prevention, health, or therapeutic treatment). In general, when a food or beverage is prepared, the compound of the present invention is added in an amount of 15 wt % or less, preferably 10 wt % or less based on the raw materials. However, for long-term intake for the purpose of health and hygiene or for the purpose of health control, the amount may be less than the above-mentioned range, and the vesicles have no problem in terms of stability, so the active ingredient may be used in an amount more than the above-mentioned range.

The type of food is not particularly limited. Examples of food to which the material may be added include meats, sausage, bread, chocolate, candies, snacks, confectioneries, pizza, instant noodles, other noodles, gums, dairy products including ice creams, various soups, beverages, tea, drinks, alcoholic beverages, vitamin complexes, and the like, and include all health functional foods in a typical sense.

The health beverage composition according to the present invention may contain various flavors or natural carbohydrates, and the like as additional ingredients as in a typical beverage. The above-described natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. As a sweetener, it is possible to use a natural sweetener such as thaumatin and stevia extract, a synthetic sweetener such as saccharin and aspartame, and the like. The proportion of the natural carbohydrates is generally about 0.01 to 0.20 g, or about 0.04 to 0.10 g per 100 ml of the composition of the present invention.

In addition to the aforementioned ingredients, the composition of the present invention may contain various nutrients, vitamins, electrolytes, flavors, colorants, pectic acids and salts thereof, alginic acid and salts thereof, organic acids, protective colloid thickeners, pH adjusters, stabilizers, preservatives, glycerin, alcohols, carbonating agents used in carbonated drinks, and the like. In addition, the composition of the present invention may contain flesh for preparing natural fruit juice, fruit juice drinks, and vegetable drinks. These ingredients may be used either alone or in combinations thereof. The proportion of these additives is not significantly important, but is generally selected within a range of 0.01 to 0.20 part by weight per 100 parts by weight of the composition of the present invention.

In this specification, the term “health functional food” is synonymous with “food for special health use (FoSHU)” and refers to foods that have been processed to efficiently exhibit bio-regulatory functions in addition to providing nutrition, demonstrating high medical and healthcare effects. Such foods may be manufactured in various forms, including tablets, capsules, powders, granules, liquids, and pills, to achieve beneficial effects in the prevention or improvement of Parkinson's disease.

The health functional food of the present invention can be prepared using conventional methods known in the field, and during its preparation, commonly used raw materials and ingredients in the industry may be added. Additionally, unlike general pharmaceuticals, health functional foods are made from food-based ingredients, offering the advantage of avoiding side effects that may occur with long-term medication use, while also being highly portable.

### MODES OF THE INVENTION

Hereinafter, preferable examples are presented to aid in the understanding of the present invention. However, these examples are provided merely for easier understanding of the present invention, and the content of the present invention is not limited by these examples.

### EXAMPLE

### Example 1. Synthesis of Isoquinoline Derivative Compound CD1-012

The isoquinoline derivative compound 2,3,5,10-Tetrahydroxy-5,6-dihydroisoquinolino[3,2-a]isoquinolin-7-iumbromide (2,3,9,10-Tetrahydroxy-5,6-dihydroisoquinolino[3,2-a]isoquinolin-7-iumbromide; CD1-012) according to the present invention was synthesized through the following process: In a dried 250 mL round flask, palmatine (1.0 g, 2.92 mmol) was dissolved in 40 mL of anhydrous CH₂Cl₂. A BBr₃ solution (12.80 mL, 12.80 mmol) was added under a nitrogen atmosphere at 0° C. to prepare the reaction mixture. The reaction mixture was stirred at room temperature for 12 hours, and the completion of the reaction was confirmed using TLC. After the completion of the reaction, 10 mL of MeOH was added to the reaction mixture and stirred for 30 minutes. It was then concentrated via a vacuum concentrator, washed with CH₂Cl₂ (100 mL×5), and dried under reduced pressure to yield a solid form of the isoquinoline derivative compound (2,3,9,10-Tetrahydroxy-5,6-dihydroisoquinolino[3,2-a]isoquinolin-7-iumbromide; CD1-012) with a yield of 99% (1.09 g, 2.98 mmol). The reaction is represented by Reaction Formula 1a below:

¹H-NMR (400 MHz, DMSO-d₆) δ10.71 (s, 1H), 10.62 (s, 1H) 9.98 (br, 1H), 9.74 (s, 1H), 9.23 (br, 1H), 8.58 (s, 1H), 7.75 (d, J=8.8 Hz, 1H) 7.61 (d, J=8.8 Hz 1H), 7.46 (s, 1H), 6.77 (s, 1H), 4.82 (t, J=6.0 Hz, 2H), 3.06 (t, J=6.0 Hz, 2H)

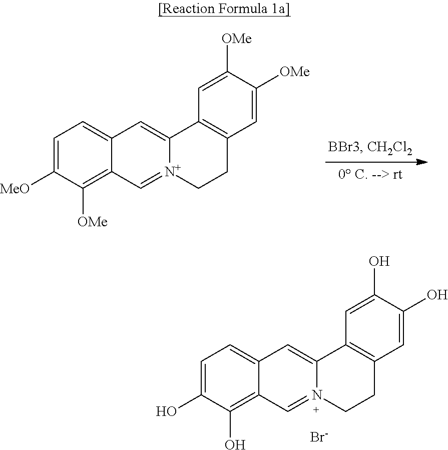

### Comparative Example 1. Carbonyl cyanide 3-chlorophenylhydrazone (CCCP)

CCCP, a representative compound promoting mitophagy, was used as Comparative Example 1.

### Comparative Example 2. Palmatine

Palmatine (CAS Number: 3486-67-7), represented by Chemical Formula 2 below, was used as Comparative Example 2.

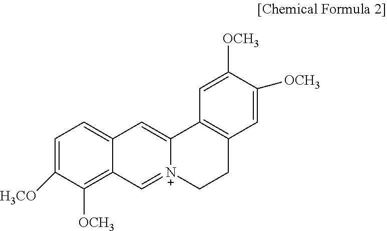 [SMILES: COc1cc2c(cc1OC)-c1cc3ccc(OC)c(OC)c3c[n+]1CC2]

### Comparative Example 3. Berberine

Berberine, represented by Chemical Formula 3 below and with CAS Numbers: 633-65-8(Berberine·HCl) or 2086-83-1(Berberine·HCl·2H₂O), was used as Comparative Example 3.

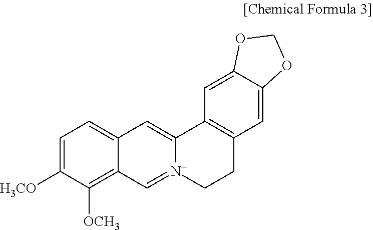 [SMILES: COc1ccc2cc3[n+](cc2c1OC)CCc1cc2c(cc1-3)OCO2]

### Example 2. Analysis of Mitophagy Activity Promoting Effect of CD1-012

### Example 2-1. Analysis of Mitophagy Activity Promoting Effect

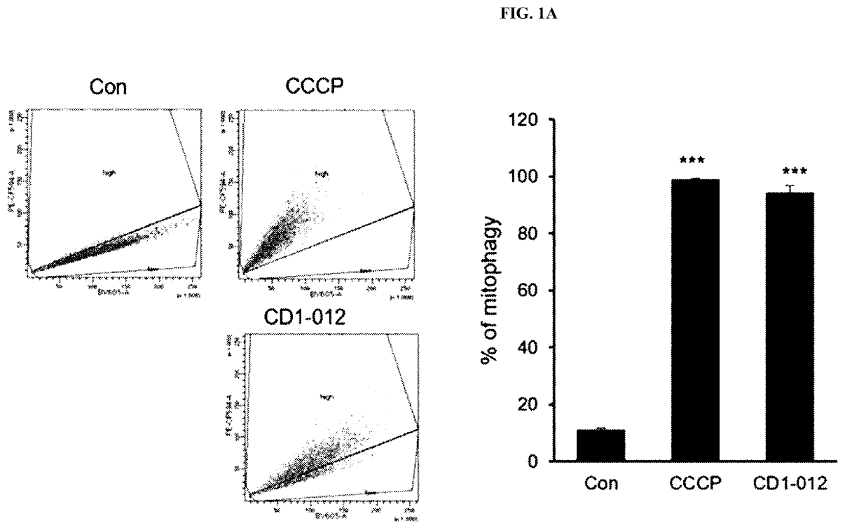

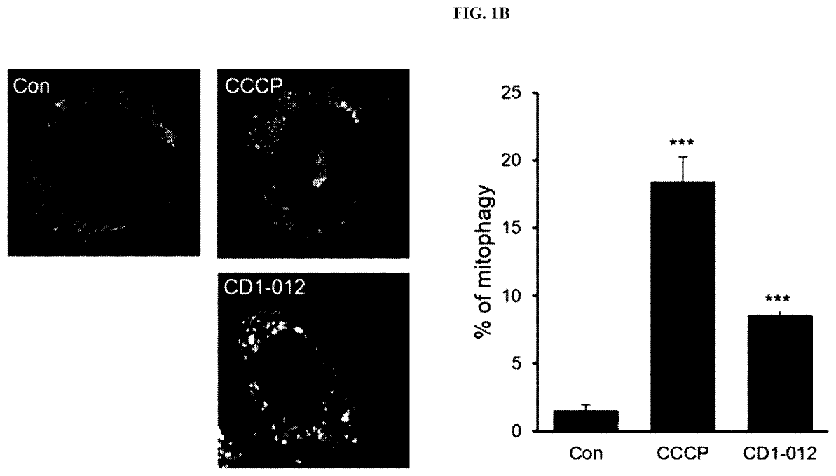

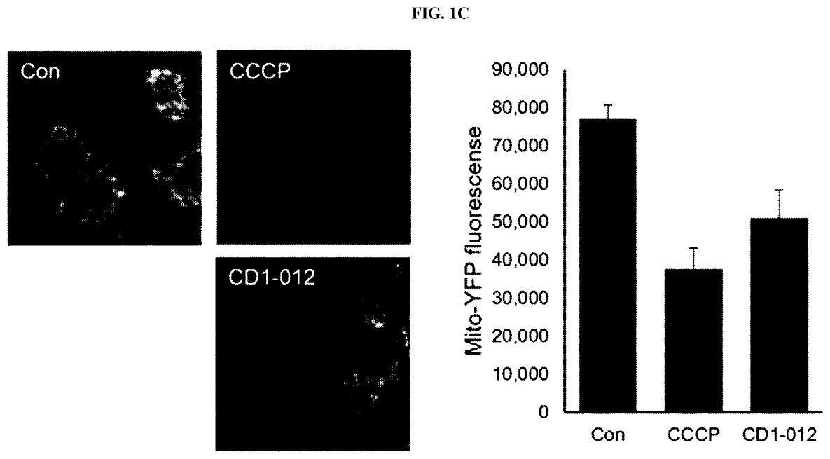

To analyze the mitophagy-promoting activity effect of the CD1-012 synthesized in the Example 1, the BEAS-2B cell line, which is a human normal lung cell line, was induced to express the mt-Keima fluorescent protein. Following this, the CD1-012 (15 μM) and the compound of Comparative Example 1, CCCP (10 μM), were treated for 24 hours. After the treatment, the mitophagy activity of each sample was measured, and the results were represented in FIGS. 1A to 1C.

Specifically, FIG. 1A represents the analysis results using flow cytometry (FACS) (treatment of CD1-012 at 15 μM for 24 hours), FIG. 1B represents the analysis results using a confocal microscope (treatment of CD1-012 at 15 μM for 18 hours), and FIG. 1C represents the measurement results of the quantitative changes in the mitochondria using a fluorescent protein, mito-YFP, which includes a targeting sequence to transport protein to the mitochondria (treatment of CD1-012 at 20 μM for 24 hours). Referring to the results, it was observed that the group treated with CD1-012 showed a significant increase in mitophagy activity compared to the untreated control group (Con). Especially in FIG. 1A, it was observed that the mitophagy activity of the sample treated with CD-012 was increased as significantly as that of the sample treated with the representative mitophagy promoting compound, CCCP.

### Example 2-2. Analysis of Mitophagy Activity Promoting Effect in Various Cell Lines

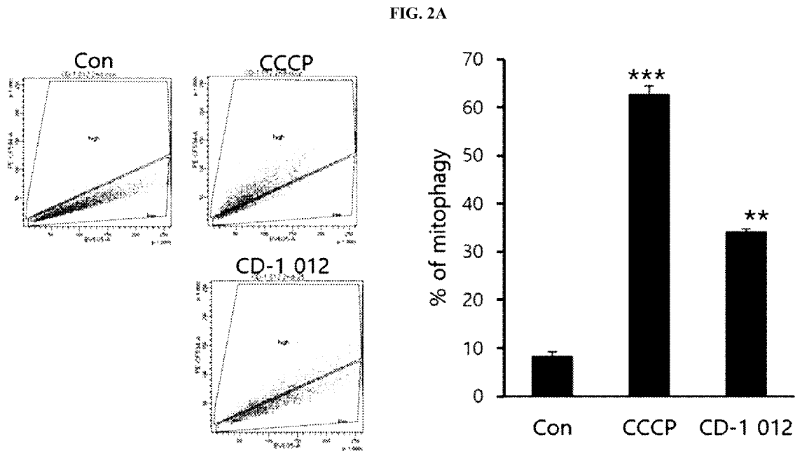

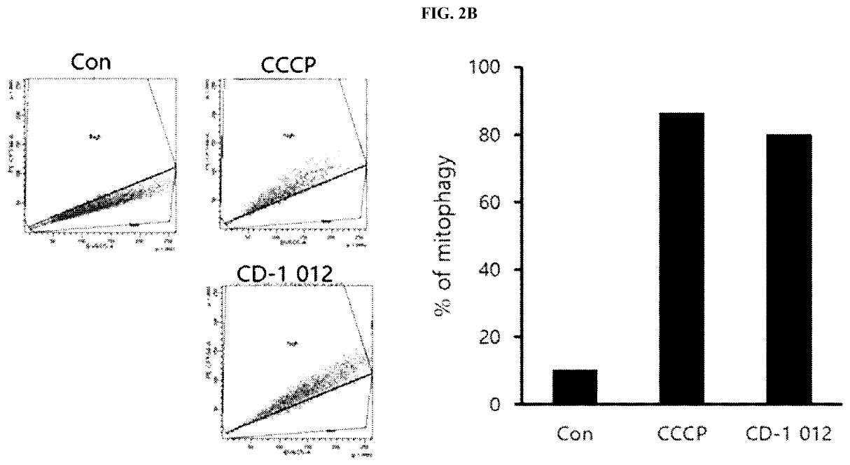

It was confirmed whether CD1-012 synthesized in Example 1 increases mitophagy activity in various cell lines. To this end, CD1-012 and Comparative Example 1 (CCCP) were treated in the SH-SY5Y cell line, a human neuroblastoma cell line expressing mt-Keima fluorescent protein, and in the HeLa-Parkin cell line, a HeLa cervical cancer cell line expressing Parkin (E3 ligase). Following this treatment, the mitophagy activity of each sample was analyzed using flow cytometry (FACS), and the results are shown in FIGS. 2A to 2B.

Specifically, FIG. 2A represents the analysis results for the SH-SY5Y cell line, and FIG. 2B represents the analysis results for the HeLa-Parkin cell line. As shown in FIGS. 2A to 2B, cells treated with the CD1-012 according to the present invention exhibited a significantly increased mitophagy activity compared to the control group (Con). These results demonstrate that the isoquinoline derivative according to the present invention can promote mitophagy activity in various cell lines.

### Example 2-3. Analysis of Mitophagy Activity Promoting Effect Depending on Treatment Concentration and Treatment Time

It was confirmed whether CD1-012 synthesized in Example 1 promotes mitophagy activity in a concentration- and time-dependent manner. To this end, the CD1-012 was treated at various concentrations or at a fixed concentration (15 μM) for different treatment durations in the BEAS-2B cell line expressing mt-Keima, and mitophagy activity was measured using a flow cytometer.

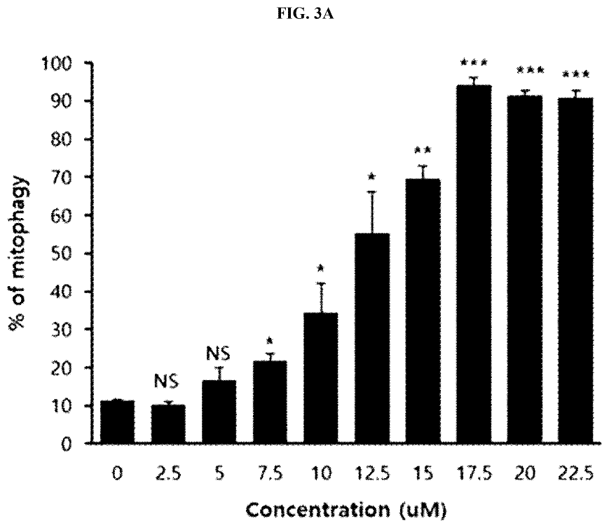

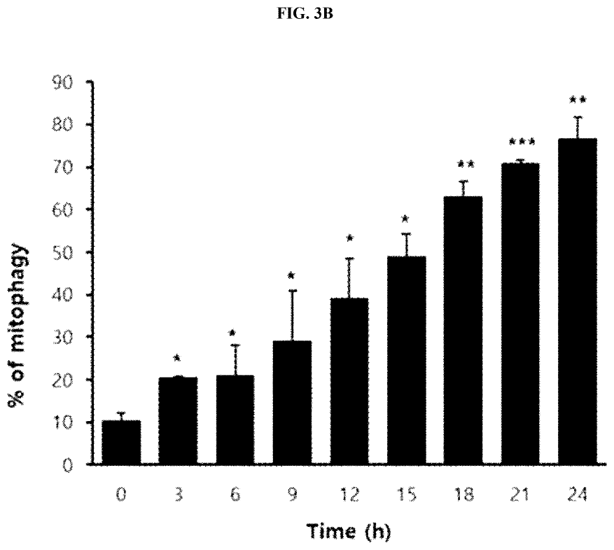

The results of mitophagy activity measurements according to the treatment concentration of CD1-012 are shown in FIG. 3A, and the results of time-dependent mitophagy activity measurements are shown in FIG. 3B. As shown in FIG. 3A, mitophagy activity began to significantly increase compared to the untreated control group when CD1-012 was treated at concentrations of 7.5 μM or higher and continued to increase in a concentration-dependent manner up to the maximum treatment concentration of 17.5 μM. Additionally, as shown in FIG. 3B, mitophagy activity began to significantly increase after 3 hours of CD1-012 treatment and reached its maximum level after 18 hours. This pattern of mitophagy activation indicates that CD1-012 directly promotes mitophagy activity in a concentration- and time-dependent manner, rather than through an indirect mechanism.

### Example 2-4. Confirmation of Specific Mitophagy Induction Activity

It was confirmed whether CD1-012 synthesized in Example 1 specifically increases only mitophagy activity. To this end, the BEAS-2B cell line expressing mt-Keima was treated with CD1-012 (15 μM) or Comparative Example 1 (CCCP, 10 μM) for 18 hours, and mitophagy activity was analyzed using a confocal microscope. Additionally, to measure autophagy activity, the BEAS-2B cell line expressing Keima fluorescent protein was incubated in Hanks' balanced salt solution (HBSS) for 3 hours to induce a starvation condition (starv.), and the results were compared with samples treated with CD1-012 (15 μM) for 18 hours using a confocal microscope.

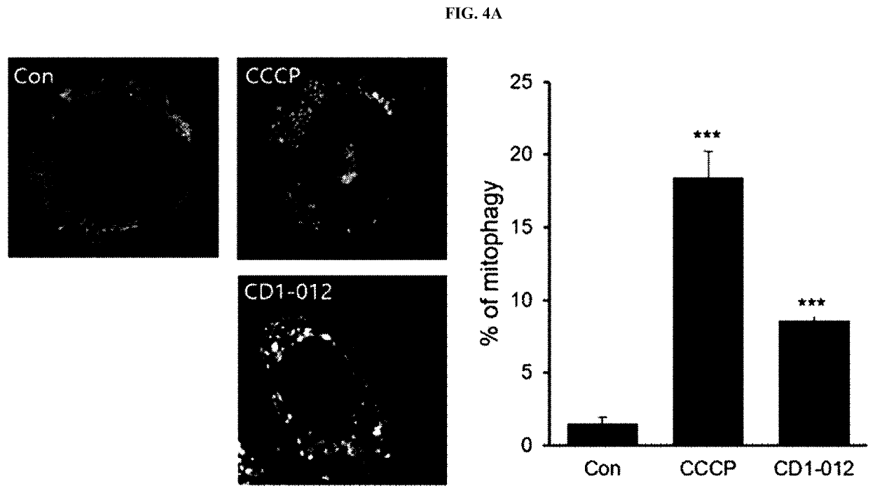

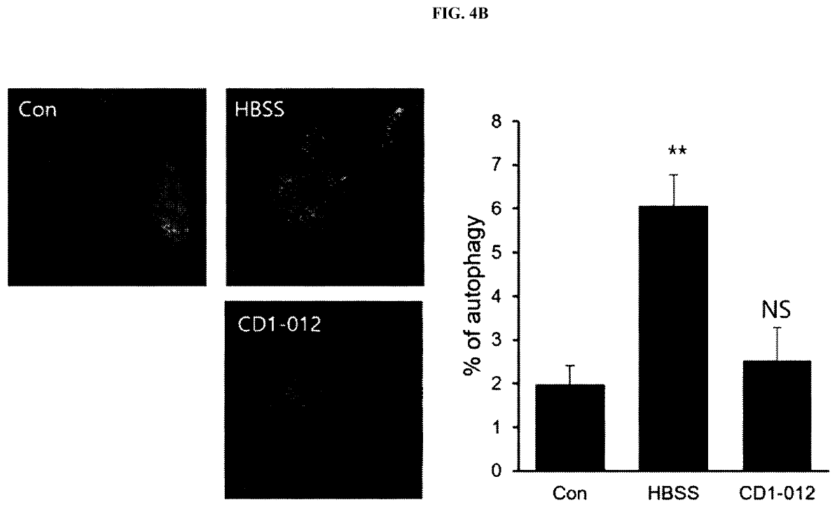

As shown in FIG. 4A, the sample treated with CD1-012 exhibited mitophagy-promoting effects similar to those observed in the CCCP-treated sample. Furthermore, as shown in FIG. 4B, the starvation-induced group exhibited a high level of autophagy, whereas the CD1-012-treated group showed no significant difference in autophagy activity compared to the untreated control group. These results suggest that the compound CD1-012 of the present invention specifically increases only mitophagy activity.

### Example 2-5. Comparison of Mitophagy Activity Induction Effects with Berberine and Palmitate

It was confirmed whether the isoquinoline derivative according to the present invention exhibits a higher mitophagy-inducing effect compared to palmatine and berberine, which are known mitophagy promoters. To this end, the human normal lung cell line BEAS-2B, expressing mt-Keima fluorescent protein, was treated with CD1-012, Comparative Example 2 (palmatine), or Comparative Example 3 (berberine) at various concentrations, and the mitophagy-inducing activity of each sample was compared.

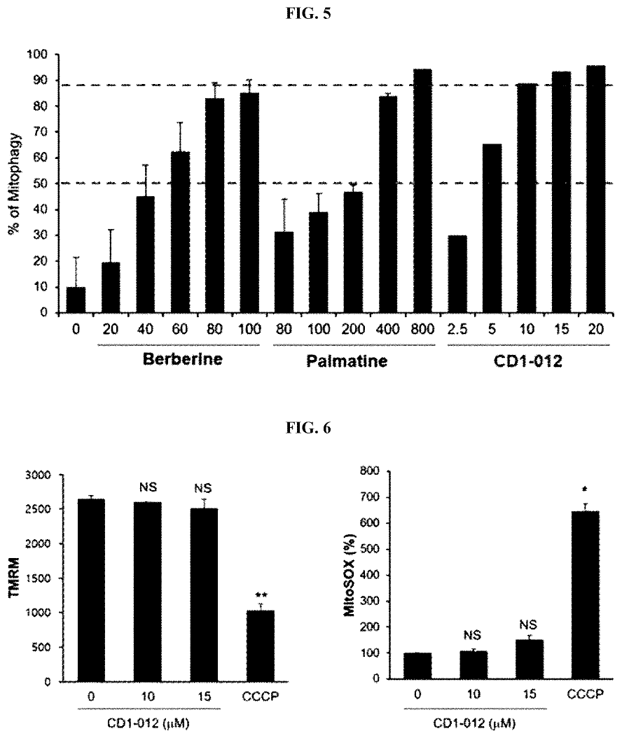

As shown in FIG. 5, mitophagy activity in BEAS-2B cells reached its maximum when treated with palmatine at 400 μM and berberine at 80 μM. However, in the CD1-012-treated group, mitophagy activity reached a comparable level at 10 μM of CD1-012 treatment. As a result, the mitophagy-inducing activity of CD1-012 was approximately 8 times higher than that of berberine and approximately 40 times higher than that of palmatine.

### Example 3. Confirmation of Induction of Mitochondrial Dysfunction of CD1-012

It was confirmed whether the isoquinoline derivative according to the present invention induces mitochondrial dysfunction, similar to CCCP. To this end, the CD1-012 (10 μM or 15 μM) and CCCP (10 μM) were treated for 24 hours, and the mitochondrial membrane potential and mitochondrial reactive oxygen species (ROS) levels in each sample were analyzed. Mitochondrial membrane potential was analyzed using the TMRM (tetramethylrhodamine methyl ester) assay, and mitochondrial ROS levels were analyzed using the MitoSOX assay.

As shown in FIG. 6, in the CCCP-treated group, mitochondrial membrane potential was significantly reduced, whereas in the CD1-012-treated group, no decrease in mitochondrial membrane potential was observed. Additionally, in the CCCP-treated group, mitochondrial ROS levels were significantly increased, while in the CD1-012-treated group, no increase in mitochondrial ROS was detected. These results indicate that, unlike CCCP, which increases mitophagy activity by inducing mitochondrial dysfunction through mitochondrial membrane potential reduction, CD1-012 does not induce mitochondrial dysfunction.

### Example 4. Confirmation of PINK1-Parkin Pathway-Independent Mitophagy Activation of mCD1-012

In this example, it was confirmed whether the mitophagy activation function of the isoquinoline derivative according to the present invention is dependent on the PINK1-Parkin pathway. To this end, after a BEAS-2B cell line, in which PINK1 was knocked down using short hairpin RNA (shRNA), was treated with CCCP (10 μM) of Comparative Example 1 or CD1-012 (15 μM) for 18 hours, the mitophagy activity of each sample was analyzed using flow cytometry (FACS).

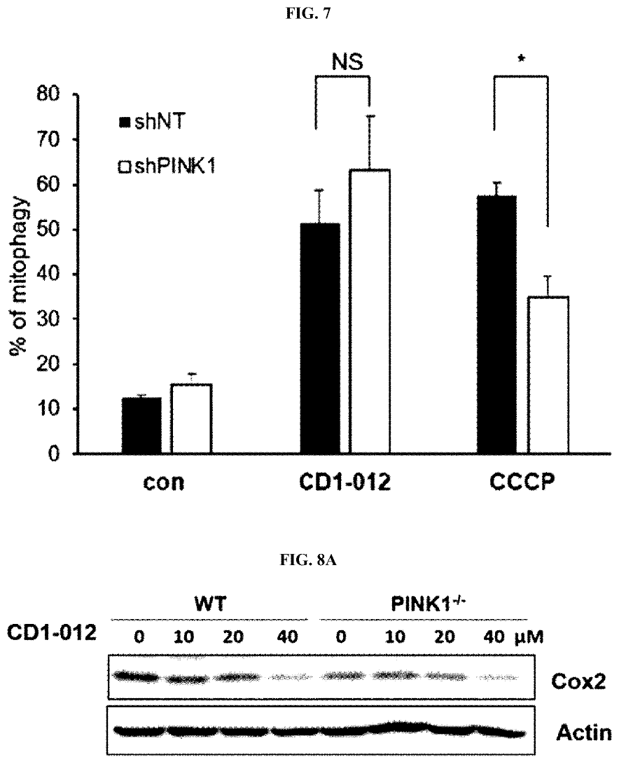

As a result, as shown in FIG. 7, the CCCP-mediated mitophagy activation in the PINK1 knockdown cell line (shPINK1) significantly decreased compared to the control cell line (shNT), whereas the CD1-012-mediated mitophagy activation did not show a significant difference compared to the untreated control. The above results demonstrate that compounds according to the present invention activate mitophagy independently of the PINK1-Parkin pathway that mediates stress-induced mitophagy.

### Example 5. Confirmation of Effect of CD1-012 on Improving Mitochondrial Dysfunction in Parkinson's Disease Cell Model

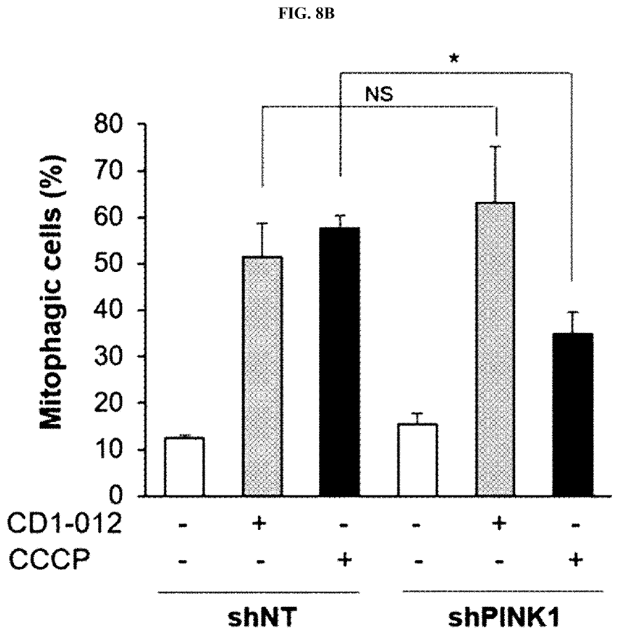

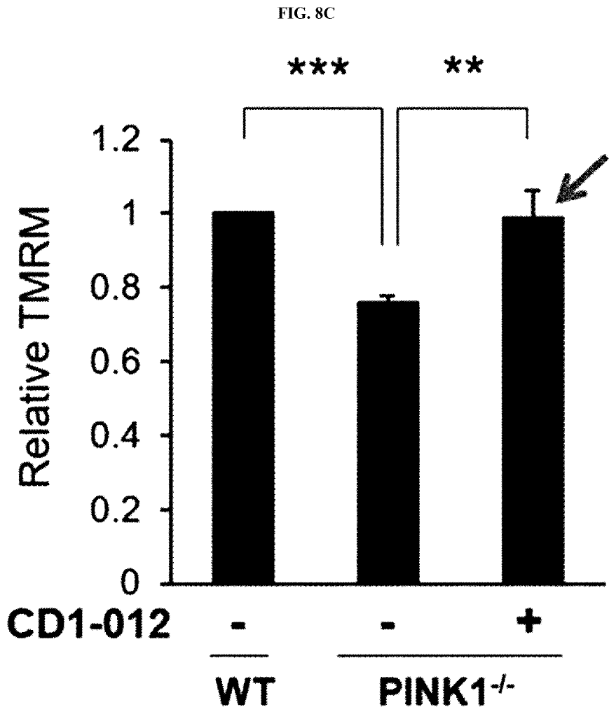

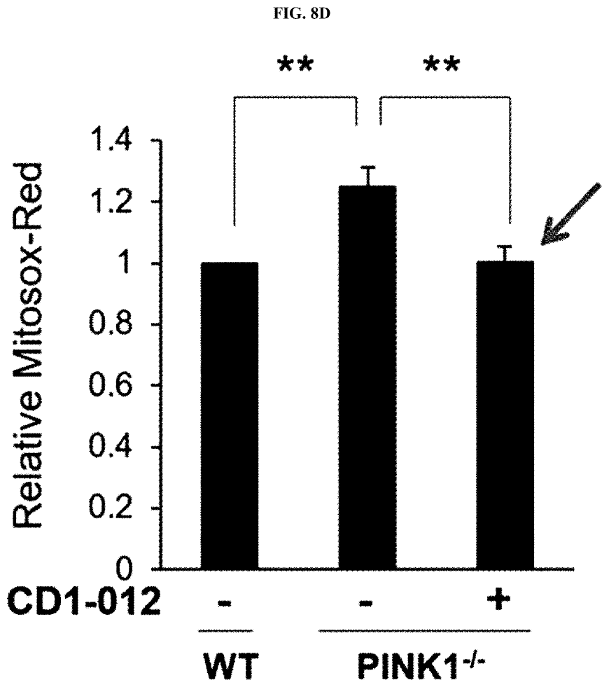

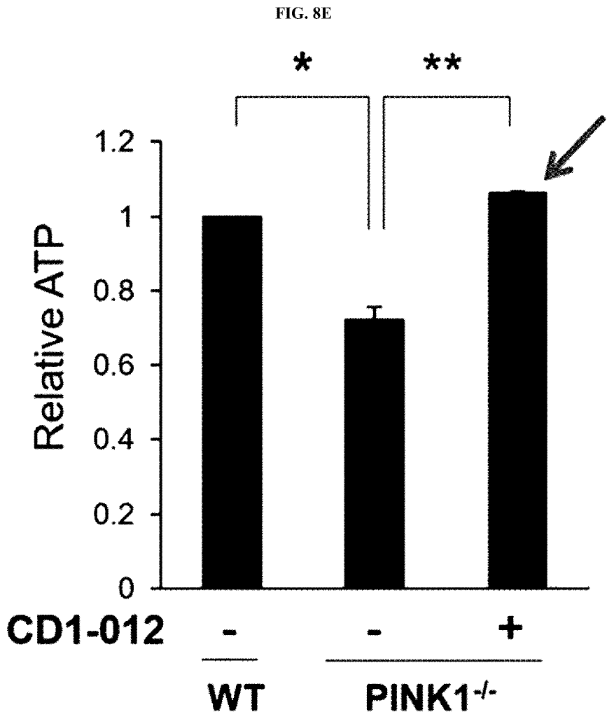

In the following example, it was confirmed whether the novel isoquinoline derivative according to the present invention has a therapeutic effect of preventing or improving Parkinson's disease. Most of all, it was confirmed whether the treatment with the isoquinoline derivative improves mitochondrial dysfunction of a Parkinson's disease cell model. To this end, first, the induction of mitophagy by CD1-012 was verified. As a result of treating wild type (WT) and PINK1-deficient (PINK1^{-/-}) mouse embryonic fibroblasts (MEFs) with CD1-012 at various concentrations for 24 hours, and measuring the amount of mitochondrial protein Cox2 by western blotting, it was confirmed that the Cox2 protein level was significantly decreased in the group treated with CD1-012 at a concentration of 40 μM (FIG. 8A). In addition, to analyze whether the mitophagy-promoting activity of CD1-012 is dependent on PINK1, PINK1 expression was inhibited (using shPNIK1) in BEAS-2B cells, which is a human normal lung cell line expressing a mitochondrial fluorescent protein, and mitophagy activity was analyzed by flow cytometry (FACS). As a result, CD1-012 (20 μM) exhibited mitophagy-promoting activity at a level similar to that of CCCP (10 μM), which is a representative mitophagy-promoting compound, and unlike CCCP, mitophagy-promoting activity was not inhibited even when PINK1 gene expression was inhibited (FIG. 8B). The above results show that the mitophagy-promoting activity of the compound according to the present invention is not dependent on PINK1.

Subsequently, PINK1-deficient MEFs were treated with CD1-012 at a concentration of 40 μM for 24 hours, and 4 days later, mitochondrial function as analyzed. The mitochondrial membrane potential was analyzed by the tetramethyl rhodamine methyl ester (TMRM) assay, and mitochondrial ROS was analyzed using the MitoSOX-Red assay. As a result, the PINK1-deficient cells had a decreased mitochondrial membrane potential compared to the wild-type cells, but the potential was restored after CD1-012 treatment (FIG. 8C). Conversely, the level of mitochondrial ROS, which was elevated compared to the wild-type cells, was reduced by CD1-012 treatment (FIG. 8D). In addition, the mitochondrial ATP production, which was reduced in PINK1-deficient cells, was also restored by CD1-012 (FIG. 8E). The above results show that the compound CD1-012 according to the present invention exhibits the effect of improving mitochondrial dysfunction in the PINK1-deficient cell line.

### Example 6. Confirmation of Effect of CD1-012 on Improving Mitochondrial Dysfunction in Drosophila Model of Parkinson's Disease

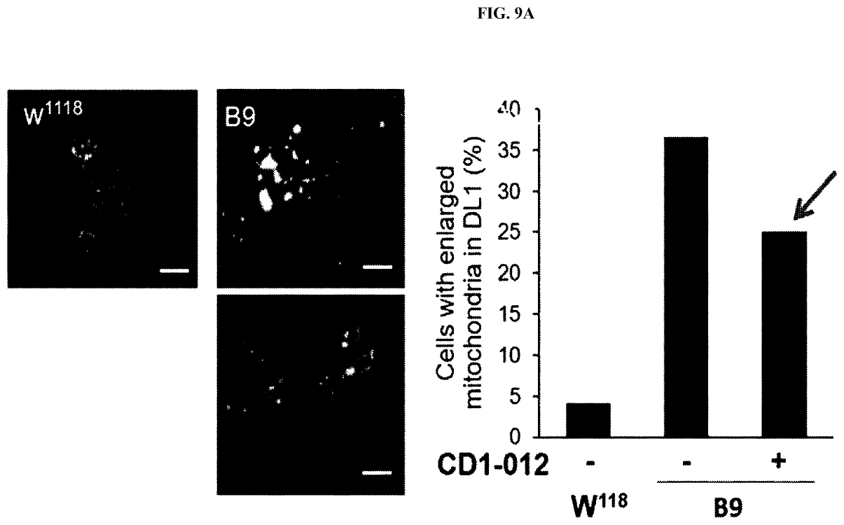

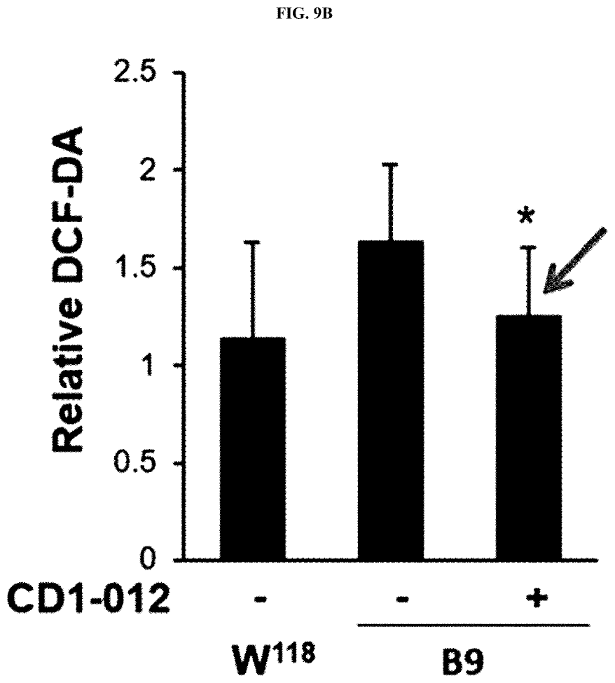

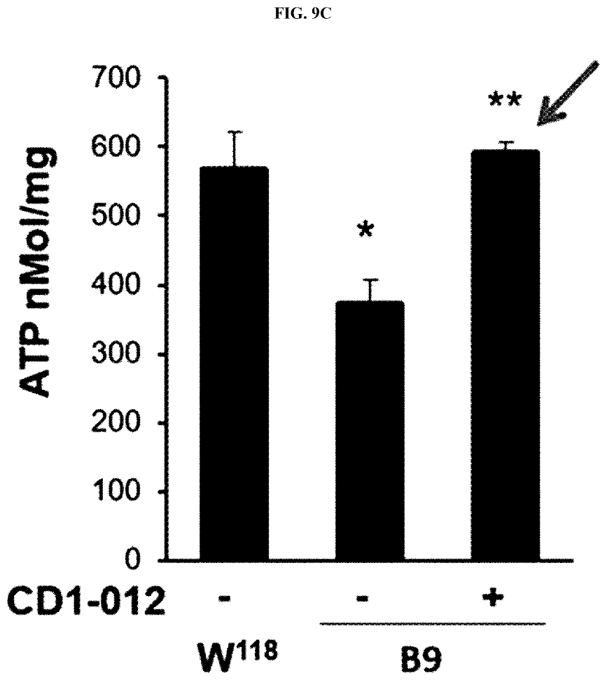

In this example, to further verify the effect of the compound of the present invention (CD1-012) on improving mitochondrial dysfunction, a Drosophila model of Parkinson's disease was used. To this end, after administering CD1-012 to PINK1 gene-deficient Drosophila, mitochondrial function was analyzed. Specifically, CD1-012 (1 mM) was administered to PINK1-deficient Drosophila (B9) for 2 weeks, and the morphology of mitochondria in the dopaminergic neurons was observed in brain tissue. As a result, the number of damaged mitochondria (with abnormally swollen morphology) observed in the PINK1-deficient Drosophila was reduced in the CD1-012-treated group as compared to an untreated control (FIG. 9A). In addition, DCF-DA staining showed that mitochondrial ROS was increased in the brain tissue of the PINK1-deficient Drosophila, and the increased ROS was significantly inhibited in the CD1-012-treated group (FIG. 9B). In addition, after 2 weeks of CD1-012 administration, it could be observed that the ATP production capacity of the brain tissue of the PINK11-deficient Drosophila was restored (FIG. 9C). These results demonstrate that CD1-012 treatment improved mitochondrial dysfunction in the Drosophila model of Parkinson's disease.

### Example 7. Confirmation of Effect of CD1-012 on Improving Mitochondrial Dysfunction in Sporadic Parkinson's Disease Cell Model

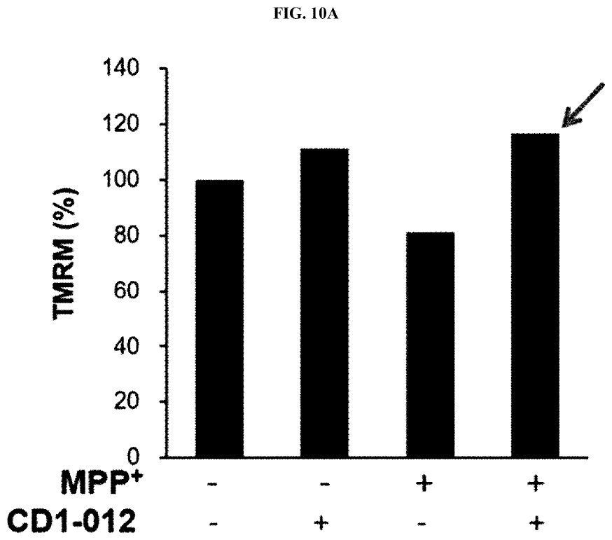

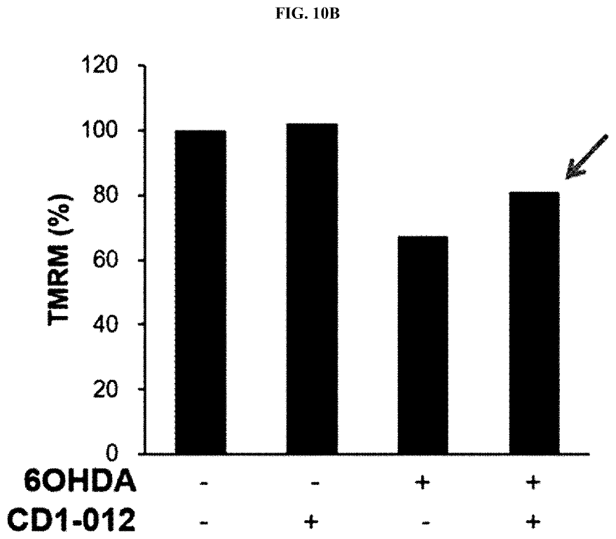

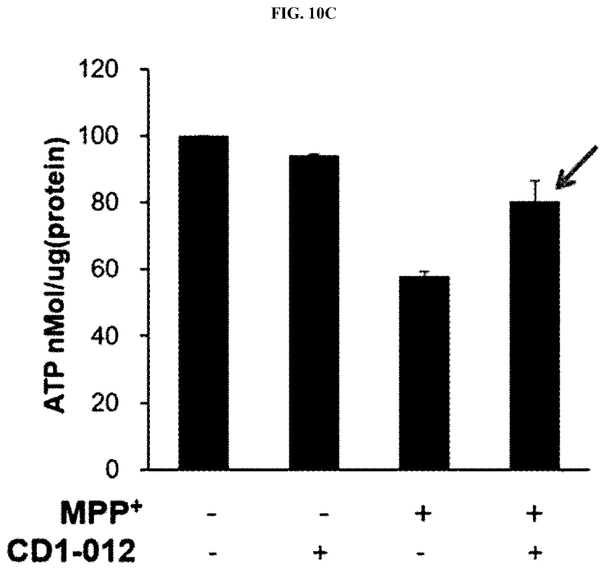

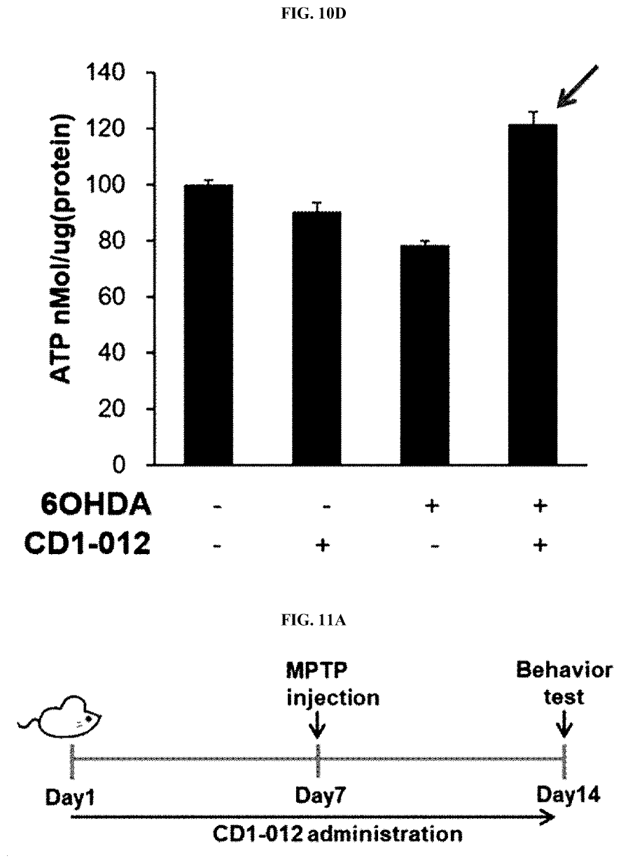

Sporadic Parkinson's disease has progressive motor/non-motor dysfunction symptoms, and most patients with Parkinson's disease have this disease. In this example, the therapeutic effect of CD1-012 on sporadic Parkinson's disease was confirmed. To this end, after a sporadic Parkinson's disease cell model was manufactured by treating a human nerve cell line H-SY5Y with the neurotoxins MPP+(62.5 μM) and 6-OHDA (40 μM) for two days, the cell model was treated with CD1-012 (20 μM) for one day and then the membrane potential and ATP production capacity of mitochondria were analyzed after two days. As a result, in the sporadic Parkinson's disease cell model, a mitochondrial membrane potential was greatly reduced by MPP⁺ and 6-OHDA and restored after the treatment with the compound of the present invention (FIGS. 10A and 10B), and the ATP synthesis capacity of mitochondria was also significantly restored after CD1-012 treatment (FIGS. 10C and 10D). The results show that the compound CD1-012 according to the present invention can also restore mitochondrial dysfunction caused by sporadic Parkinson's disease through its mitophagy-promoting activity.

### Example 8. Confirmation of Effect of Treating Behavioral Abnormalities in Parkinson's Disease Animal Model by CD1-012

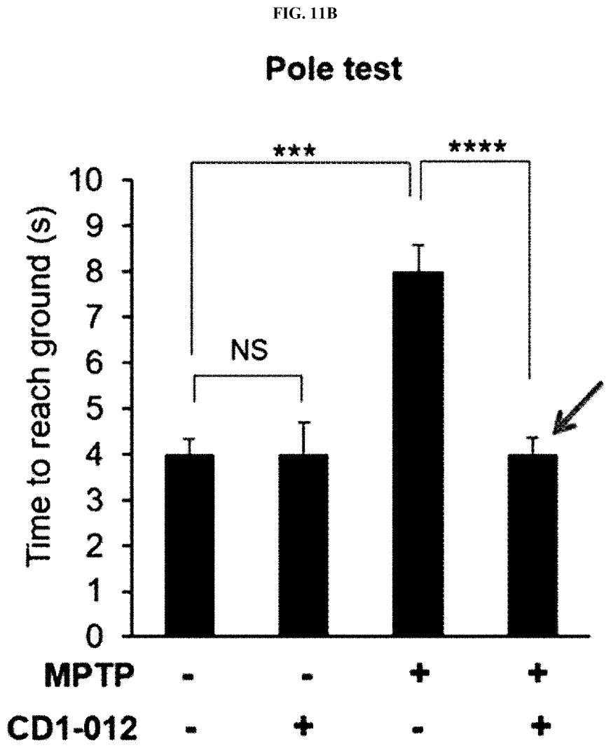

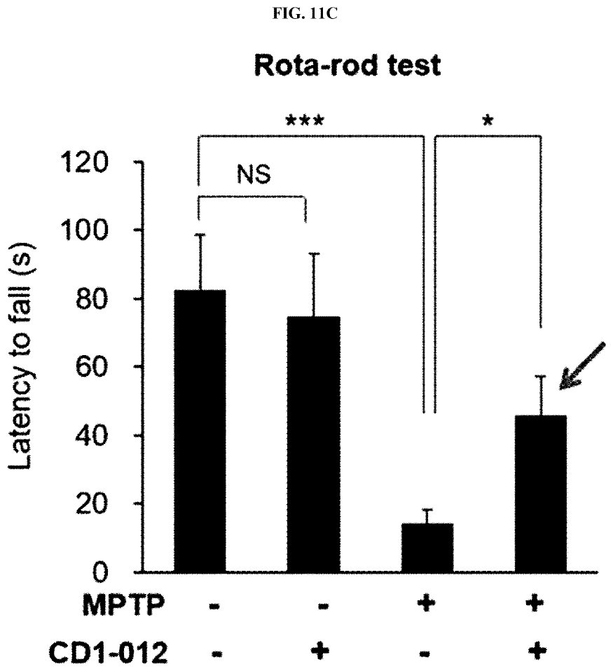

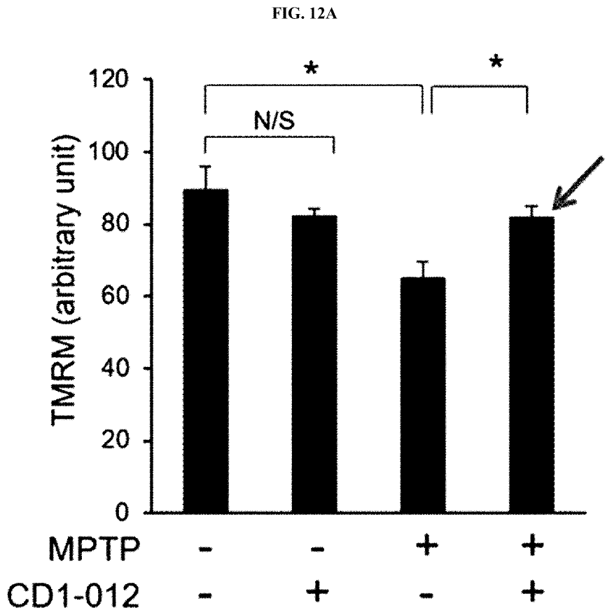

In this example, the effect of the compound of the present invention (CD1-012) on treating Parkinson's disease was confirmed using a mouse model of Parkinson's disease. To this end, a mouse model of Parkinson's disease established by injecting the neurotoxin, 1-methyl-4-phenyl-1,2,3,6-tetrahydropyridine (MPTP), was used. After nasal administration of CD1-012 at a concentration of 1 mg/kg daily for 2 weeks to the mouse model, motor performance was analyzed using the pole test and Rota-rod test (FIG. 11A). First, the pole test showed that, compared to the normal control, the Parkinson's disease model injected with MPTP had a significantly increased time to reach the ground, whereas in the CD1-012-administered mouse, motor performance recovered to a level equivalent to that of the normal control (FIG. 11B). In addition, the Rota-rod test can show that the time spent on the cylinder was significantly reduced in the Parkinson's disease model compared to the normal control, but was markedly recovered in CD1-012-administered mouse (FIG. 11C). Particularly, it can be seen that normal mice administered CD1-012 alone showed no significant difference in motor performance compared to the untreated normal control, indicating no toxicity of CD1-012 itself. The results demonstrate that the compound CD1-012 of the present invention can improve and treat impaired motor performance caused by Parkinson's disease.

### Example 9. Confirmation of Effects of Improving Mitochondrial Dysfunction in Parkinson's Disease Animal Model and Inhibiting Death of Dopaminergic Neurons by CD1-012

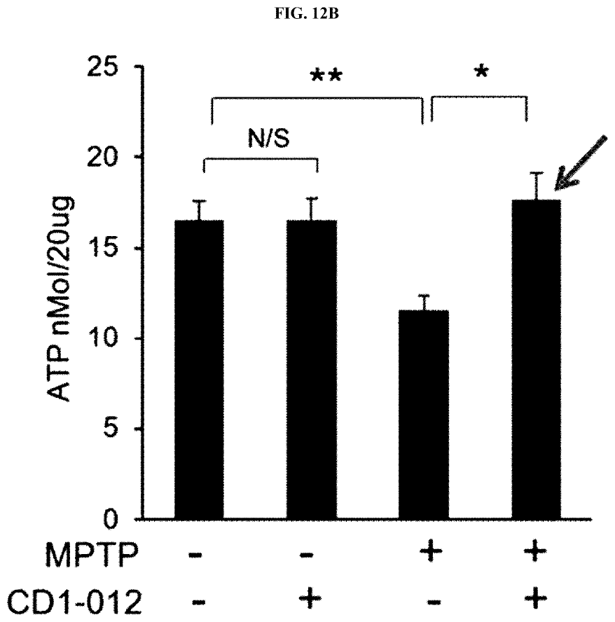

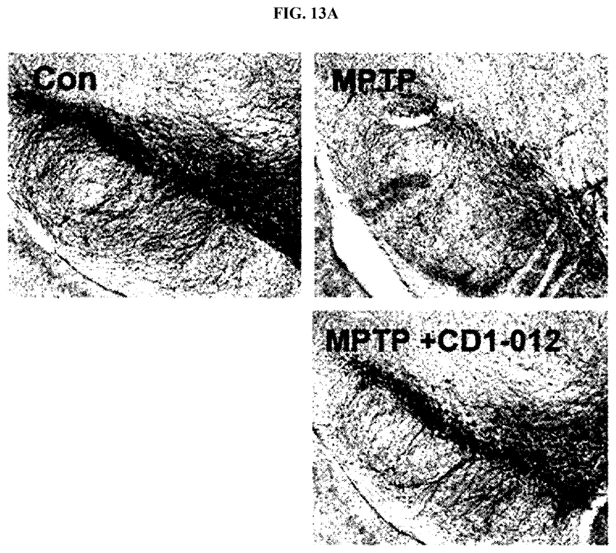

Subsequently, it was confirmed that the compound CD1-012 of the present invention has an effect of improving mitochondrial dysfunction using an animal model of Parkinson's disease. To this end, after separating mitochondria from the substantia nigra of the mice undergoing the behavioral experiment in Example 4, the membrane potential of mitochondria was analyzed through TMRM (100 nM, 37° C., 30 min) fluorescent staining, and the mitochondrial function was analyzed by measuring ATP production (analyzed for 5 mice per group). As a result, compared to the control, the Parkinson's disease model mouse group administered the neurotoxin MPTP showed a decrease in membrane potential of mitochondria and a significant decrease in ATP synthesis capacity, whereas the CD1-012-coadministered group showed a restoration in the membrane potential and the ATP synthesis capacity in mitochondria compared to the normal control (FIGS. 12A and 12B). The above results show that the compound CD1-012 of the present invention can improve mitochondrial dysfunction in the animal model of Parkinson's disease.

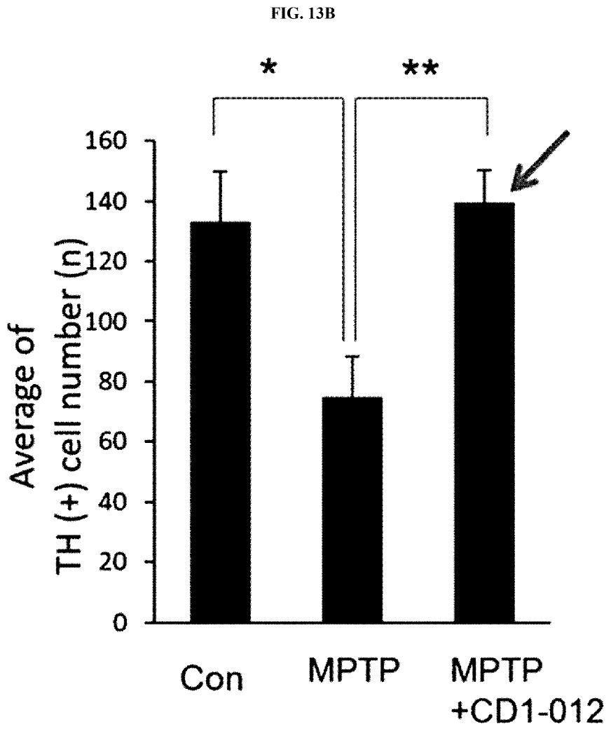

Subsequently, it was confirmed whether CD1-012 administration can inhibit dopaminergic neuron death in the substantia nigra of the animal model of Parkinson's disease. To this end, the number of dopaminergic neurons was measured by performing TH immunochemical staining on the substantia nigra of the mice undergoing the behavioral experiment in Example 4. As a result, the number of dopaminergic neurons was reduced by MPTP to almost half of the normal control in the animal model of Parkinson's disease, whereas the number of dopaminergic neurons was restored to a level similar to the control in the CD1-012-administered group (FIGS. 13A and 13B). These results demonstrate that the compound CD1-012 of the present invention can inhibit dopaminergic neuron death, which is the underlying cause of Parkinson's disease, by restoring mitochondrial function.

As described in the above examples, the present inventors confirmed that the isoquinoline compound CD1-012 with mitophagy-specific promoting activity can improve mitochondrial dysfunction in hereditary and sporadic Parkinson's disease models. The effect of CD1-012 on improving mitochondrial function was also verified in the Parkinson's disease mouse model, and in particular, it was confirmed through the specific experiments that the compound can improve behavioral abnormalities caused by Parkinson's disease and inhibit dopaminergic neuron death. Therefore, the compound according to the present invention is a material that enables the fundamental treatment of Parkinson's disease and is expected to be useful in preventing and treating Parkinson's disease.

### Manufacturing Example of Drug

The active material according to the present invention may be formulated in various forms depending on its purpose. The following are some methods of formulating the active material according to the present invention comprising the active ingredient, and the present invention is not limited thereto.

### <Manufacturing Example of Drug 1> Manufacturing of Acid Formulation

Active material 2 g
Lactose 1 g

The ingredients were mixed and filled in a sealed pack to prepare the acid formulation.

### <Manufacturing Example of Drug 2> Manufacturing of Tablet

Active material 100 mg
Cornstarch 100 mg
Lactose 100 mg
Magnesium stearate 2 mg

The ingredients were mixed and then tableted according to conventional tablet manufacturing methods to prepare the tablet.

### <Manufacturing Example of Drug 3> Manufacturing of Capsule

Active material 100 mg
Cornstarch 100 mg
Lactose 100 mg
Magnesium stearate 2 mg

The ingredients were mixed and then filled into gelatin capsules according to conventional capsule manufacturing methods to prepare the capsule formulation.

### <Manufacturing Example of Drug 4> Manufacturing of Injectable

Active material 10 μg/mL
Diluted hydrochloric acid BP until pH 3.5
Injectable sodium chloride BP up to 1 mL

The active material according to the present invention was dissolved in an appropriate volume of injectable sodium chloride BP, and the pH of the resulting solution was adjusted to pH 3.5 using diluted hydrochloric acid BP. The volume was adjusted using injectable sodium chloride BP and mixed thoroughly. The solution was filled into transparent glass 5 mL type I ampoules, sealed under an air upper grid by melting the glass, and sterilized by autoclaving at 120° C. for at least 15 minutes to prepare the injectable.

### <Manufacturing Example of Drug 5> Manufacturing of Nasal Spray

Active material 1.0 g
Sodium acetate 0.3 g
Methylparaben 0.1 g
Propylparaben 0.02 g
Sodium chloride appropriate amount
HCl or NaOH for pH adjustment appropriate amount
Purified water appropriate amount

According to conventional nasal spray manufacturing methods, it was prepared to contain 3 mg of active material per 1 mL of saline (0.9% NaCl, w/v, solvent is purified water). It was filled into an opaque spray container and sterilized to prepare the nasal spray.

### <Manufacturing Example of Drug 6> Manufacturing of Liquid Formulation

Active material 100 mg
High-fructose corn syrup 10 g
Mannitol 5 g
Purified water appropriate amount

Each ingredient was dissolved in purified water according to conventional liquid formulation manufacturing methods, lemon flavor was added, and then the ingredients were mixed. Purified water was added to adjust the total to 100 mL, and it was filled into a brown bottle and sterilized to prepare the liquid formulation.

### Manufacturing Example of Health Food

The active material according to the present invention may be manufactured into various forms of health food depending on its purpose. The following are examples of manufacturing health foods comprising an active material as an active ingredient according to the present invention, but the present invention is not limited thereto.

### <Manufacturing Example of Health Food 1> Manufacturing of Dairy Products

0.01-1 weight part of the active material of the present invention was added to milk, and various dairy products such as butter and ice cream were manufactured using the milk.

### <Manufacturing Example of Health Food 2> Manufacturing of Seonsik

Brown rice, barley, glutinous rice, and barnyard millet were gelatinized by a known method and dried, and then mixed and ground into a powder of 60 mesh. Black beans, black sesame, and perilla seeds were also steamed by a known method, dried, and then mixed and ground into a powder of 60 mesh. The active material of the present invention was concentrated under reduced pressure in a vacuum concentrator, and a dried powder was obtained. The following proportions of the manufactured cereals, seeds, and dried powder of the active material were mixed.
Cereals (brown rice 34 weight parts, barnyard millet 19 weight parts, barley 20 weight parts),
Seeds (perilla seeds 7 weight parts, black beans 8 weight parts, black sesame 7 weight parts),
Active material (2 weight parts),
Ganoderma lucidum (1.5 weight parts), and
Rehmannia glutinosa (1.5 weight parts).

### Manufacturing Example of Health Functional Food Composition

Active materials according to the present invention may be manufactured into various forms of health functional food compositions depending on its purpose. The following are examples of manufacturing health functional food compositions comprising an active material as an active ingredient according to the present invention, but the present invention is not limited thereto.

### <Manufacturing Example of Health Functional Food Composition 1> Manufacturing of Health Functional Food Composition

Active material 100 mg
Vitamin mixture appropriate amount
Vitamin A acetate 70 μg
Vitamin E 1.0 mg
Vitamin B1 0.13 mg
Vitamin B2 0.15 mg
Vitamin B6 0.5 mg
Vitamin B12 0.2 μg
Vitamin C 10 mg
Biotin 10 μg
Nicotinamide 1.7 mg
Folic acid 50 μg
Pantothenic acid calcium 0.5 mg
Mineral mixture appropriate amount
Ferrous sulfate 1.75 mg
Zinc oxide 0.82 mg
Magnesium carbonate 25.3 mg
Potassium dihydrogen phosphate 15 mg
Calcium hydrogen phosphate 55 mg
Potassium citrate 90 mg
Calcium carbonate 100 mg
Magnesium chloride 24.8 mg

The composition ratio of the vitamins and minerals mixture was mixed as a desirable example suitable for relatively health functional foods, but it is acceptable to modify the mixing ratio at will. The above ingredients were mixed and then granulated, and may be used to manufacture the health functional food composition according to a conventional method.

### <Manufacturing Example of Health Functional Food Composition 2> Manufacturing of Health Functional Beverage

Active material 100 mg
Citric acid 100 mg
Oligosaccharide 100 mg
Plum concentrate 2 mg
Taurine 100 mg
Add purified water to make a total of 500 mL

The above ingredients were mixed and then heated with stirring at 85° C. for about 1 hour. The resulting solution was filtered, filled into a sterilized container, and sterilized by sealing. After refrigeration, it may be used to manufacture the health beverage composition of the present invention. The composition ratio was mixed as a desirable example suitable for relatively preferred beverages, but it may be modified according to regional and ethnic preferences, demand groups, demand countries, and usage purposes.

The foregoing description of the present invention is provided by way of example, and those skilled in the art to which the present invention pertains will understand that various modifications can be easily made in other specific forms without departing from the technical spirit or essential characteristics of the present invention. Therefore, the embodiments described above should be understood as illustrative in all respects and not as limiting.

### INDUSTRIAL APPLICABILITY

The present invention relates to a pharmaceutical composition for preventing or treating Parkinson's disease, and was completed by confirming that an isoquinoline derivative discovered through screening based on mitophagy activity exhibits excellent mitophagy-promoting effects, and thus can be utilized as a fundamental treatment for Parkinson's disease. Specifically, it was confirmed that the isoquinoline derivative according to the present invention can not only improve mitochondrial dysfunction closely related to Parkinson's disease in an animal model of Parkinson's disease, but also improve impaired motor function in an animal model of Parkinson's disease. Particularly, in the animal model of Parkinson's disease treated with the isoquinoline derivative according to the present invention, dopaminergic neuron death, which is the main cause of Parkinson's disease, appears to be effectively inhibited. Therefore, the isoquinoline derivative according to the present invention is expected to be useful in the fields of prevention, improvement, and/or treatment of Parkinson's disease as a fundamental therapeutic agent for the disease.

1. A method of preventing or treating Parkinson's disease comprising: administering a pharmaceutical composition comprising an isoquinoline derivative represented by Chemical Formula 1 below or a pharmaceutically acceptable salt thereof as an active ingredient to a subject in need thereof

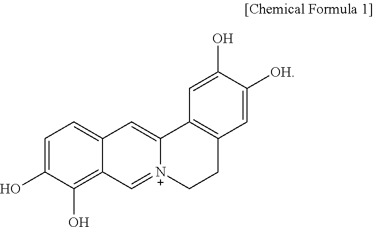 [SMILES: Oc1cc2c(cc1O)-c1cc3ccc(O)c(O)c3c[n+]1CC2]

2. The method of claim 1, wherein the isoquinoline derivative or pharmaceutically acceptable salt thereof specifically promotes mitophagy activity.

3. The method of claim 2, wherein the mitophagy activity is independent of PINK1.

4. The method of claim 1, wherein the isoquinoline derivative or pharmaceutically acceptable salt thereof increases the number or activity of dopaminergic neurons.

5. The method of claim 1, wherein the Parkinson's disease is hereditary Parkinson's disease or sporadic Parkinson's disease.

6. The method of claim 1, wherein the isoquinoline derivative or pharmaceutically acceptable salt thereof is present in an amount of greater than 0 to 40 μM.

7. The method of claim 1, wherein the isoquinoline derivative or pharmaceutically acceptable salt thereof improves mitochondrial dysfunction.

8. The method of claim 7, wherein the improvement in mitochondrial dysfunction is one selected from the group consisting of:
(a) maintaining the normal membrane potential level in mitochondria;
(b) reducing the reactive oxygen species (ROS) level in mitochondria; and
(c) increasing the ATP synthesis capacity of mitochondria.

9. The method of any one of claims 1-8, wherein the method comprises using a kit comprising the pharmaceutical composition and instructions.

10. A method of preventing or improving Parkinson's disease comprising administering a food composition comprising an isoquinoline derivative represented by Chemical Formula 1 below or a sitologically acceptable salt thereof as an active ingredient to a subject in need thereof

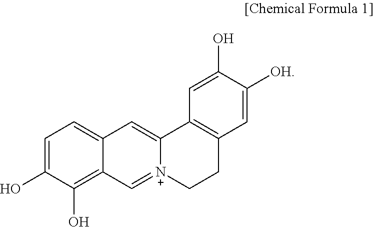 [SMILES: Oc1cc2c(cc1O)-c1cc3ccc(O)c(O)c3c[n+]1CC2]

11. The method of claim 10, wherein the Parkinson's disease is hereditary Parkinson's disease or sporadic Parkinson's disease.

12-14. (canceled)
